(19) United States
(12) Patent Application Publication
(10) Pub. No.: US 2014/0357718 A1
(43) Pub. Date: Dec. 4, 2014

(54) COMPOSITIONS FOR USE IN CONTROLLING INTESTINAL MICROFLORA

(71) Applicant: BASF SE, Ludwigshafen (DE)

(72) Inventors: Dieter Feuerstein, Dudenhofen (DE); Ulrich Roser, Romerberg (DE); Nicolas Vautravers, Mannheim (DE); Gerd Diebold, Reutlingen (DE)

(73) Assignee: BASF SE, Ludwigshafen (DE)

(21) Appl. No.: 14/292,981

(22) Filed: Jun. 2, 2014

Related U.S. Application Data
(60) Provisional application No. 61/830,182, filed on Jun. 3, 2013.

(51) Int. Cl. A61K 31/20; A23K 1/16; A61K 31/19

(52) U.S. Cl. CPC A61K 31/20; A61K 31/19; A23K 1/1609
USPC 514/558

(57) ABSTRACT

The present invention relates to compositions containing organic acids and emulsifiers and their use in controlling intestinal microflora in production animals. In particular, the present invention relates to the use of such compositions for reducing foodborne pathogens.

The present invention relates to compositions containing organic acids and emulsifiers and their use in controlling intestinal microflora in production animals. In particular, the present invention relates to the use of such compositions for reducing foodborne pathogens in poultry.

Resistance of bacterial pathogens against antibiotics has become a worldwide problem for the treatment of infections. The supplementation of animal feed with antibiotics to increase production has further contributed to promote the resistance of foodborne pathogens which are dangerous to human consumers.

Since the ban of antimicrobial growth promoters in the European Union, feed manufacturers have been actively looking for alternatives for the control of intestinal microflora in production animals. Especially important for the present situation in the EU is the control of the foodborne pathogens Campylobacter jejuni and Salmonella enterica. Future legislation has been proposed to set strict limits for their incidence. Unless new alternatives for the control of foodborne pathogens are developed soon, however, the regulations remain unrealistic to be met.

An overview of different strategies to control Camphylobacter contamination of poultry meat is given by Pasquali et al. in the World's Poultry Science Journal, Vol. 67, March 2011. Campylobacteriosis is reported to be one of the most common foodborne diseases in Europe having had an incidence of 190,566 cases of human infection in 2008. Contamination with Camphylobacter may occur at different stages of broiler meat production, finally leading to a transmission to humans. Important goals to prevent circulation of the pathogen among animals are enforcement of biosecurity measures on the poultry farms and efficient litter management. Among the most common pre-harvest strategies to reduce Camphylobacter contamination are the administration of antibiotics, probiotics and vaccines as well as the supplementation of feed and drinking water with Camphylobacter inhibiting agents.

In order to implement measures against transmission of pathogens and prevent further promotion of resistance, antimicrobially active compounds are needed which are toxicologically safe and not easily subject to resistance.

Ever since it has been recognized that antibiotics should not be used anymore in large scale animal production, the search for alternative pathogen inhibiting compounds has become increasingly important to the food industry. Organic acids, in particular naturally occurring fatty acids, as well as their esters and glycerides are promising candidates to fulfill the above mentioned requirements. The antimicrobial properties of these compounds have been frequently reported in the literature and the mechanisms for their pathogen inhibiting actions are subject to intense research.

Organic acids of various chain lengths are used to combat Salmonella spp. in poultry and they show species specific antibacterial effects as reviewed by Van Immerseel et al. in Avian Pathology, 2006, 35(3), 182-188. Medium chain fatty acids and proprionate are believed to influence epithelial cell invasion by Salmonella spp. by affecting expression levels of so-called invasion genes.

The effect of feed supplementation with medium chain fatty acids on the susceptibility of broilers for colonization by Camphylobacter was studied by Van Gerwe et al. (Veterinary Microbiology (2008), doi:10.1016/j.vetmic.2009.11.029) who investigated mixtures of C₈-C₁₂ fatty acids for their anti-Camphylobacter activity in acidified feed. The results suggest that a feed supplementation with medium chain fatty acids decreases the susceptibility for Camphylobacter colonization though the mechanism of anti-Camphylobacter activity remains to be elucidated.

Glycerol monolaurate (monolaurin), the glycerol ester of lauric acid, is generally recognized for its strong antimicrobial properties. A review of the antiviral and antibacterial activity of monolaurin and lauric acid is given by Lieberman et al. in Alternative and Complementary Therapies, 2006, 12(6), 310-314. While monolaurin is still many times more antimicrobially active than lauric acid, lauric acid (C12) exhibits stronger antibacterial properties than other medium chain triglycerides such as caprylic acid (C8), capric acid (C10) or myristic acid (C14). Research suggests that these compounds can act as surfactants by solubilizing the lipids and phospholipids of a pathogen membrane.

Glycerol monolaurate has also been reported to inhibit effects of gram positive bacterial exotoxins on mammalian cells as described by Peterson et al. in Biochemistry, 2006, 45(7), 2387-2397. Mechanisms to counteract cytotoxicity of these agents appear to be inhibition of signal transduction in the bacterial cells thereby preventing the production of gram positive exotoxins as well as the ability to inhibit immunoproliferative and immunstimmulatory effects of certain exotoxins (superantigens).

Similar effects of glycerol monolaurate, i.e. inhibition of cell signaling, have been found to be a promising option to prevent immunodeficiency virus transmission by Li et al. (Nature, 2009, 458, 1034-1038).

The application of monolauric acid with glycerol monolaurate as antimicrobial agents in pig feed to prevent circulation of bacteria, especially Streptococcus, was presented by De Snoeck in an oral session of the 22nd International Pig Veterinary Society Congess on Jun. 12, 2012 in Jeju, Korea.

Antimicrobial effects against a number of pathogenic bacteria and viruses are described for fatty acids and monoglycerides from natural lipids, with monocaprin being the most efficient one, by Thormar et al. (Recent Developments in Antiviral Research, 2001, 1, 157-173). It is noted that pathogens strongly differ in their sensitivities to individual lipids while the lipids also show varying activities against each pathogen.

In summary, antimicrobial properties and mechanisms to be considered when using organic acids and their (glycerol) esters to control bacterial growth seem at least to include acidity, surfactant/solubilizing effects as well as interference with cellular signaling and gene regulation.

Due to the variety of the nature of antimicrobial actions, it becomes very difficult to predict how efficient a compound will be in a certain application environment. It would be desirable to take advantage of all of the above mentioned actions by combining different agents in one composition. Such a combination, however, further increases complexity and unpredictability as well as a risk of undesirable interactions between the individual components. In addition, species specific effects, the causes of which are still under investigation, have to be taken into account when designing a composition. In this context, it would also be useful to be able to ensure survival of commensal intestine bacteria, which might be beneficial to animal health, ideally providing them with a competitive advantage over the pathogens.

A primary object of the present invention was therefore to provide a composition which is particularly suitable to control intestinal microflora in production animals.

A wide range of acids could potentially be used to control intestinal pathogens, individually or in combination. Medium chain fatty acids are especially interesting since their pKa values are close to 5 and they are relatively lipophilic, properties expected to contribute to efficiency for the application in biological systems operating at pH close to neutrality. Acid and emulsifier combinations can have synergistic effects which cannot be predicted. Therefore, the only way to design a suitable composition is to test how various acids and combinations at various concentrations affect the growth of the selected indicator bacteria in a simple biological system such as a pure bacterial culture.

A fundamentally relevant factor for the efficiency of antimicrobial action is the pH. When an acid composition is applied on feed, it will decrease the matrix pH to the extent determined by the strength of the specific acid and its concentration. Since acid compositions are usually acting against bacteria more effectively at the pH close to or below their pKa value, it is to be expected that when the system is well buffered to neutral pH, acidic products are less inhibitory. Independent of the acid inclusion in the diet, feed digest becomes acidified in the proventriculus of a chicken but when the digesta enters the duodenum, neutralising pancreatine will be mixed in and the pH will reach 6.5 whether or not the diet was initially acidified. Therefore, when the acid reaches the small intestine the bacteria present will be exposed to it at pH 6.5-7.0. This means that to be effective also in the small intestine, in-feed acids should be antibacterial also in dissociated form. It is worth noting that the stronger the acid the smaller proportion is protonated at neutral pH; protonated form penetrates better through cell membrane and is, therefore, more inhibitory. Consequently, it is to be expected that at equal molar concentration, weaker acids would be more powerful growth inhibitors.

To mimic both situations, it is important to test the effect of acids under two different conditions. In practice, the test mimicking feed preservation application needs to be carried out in a medium, in which the pH can shift freely when acids at various concentrations are added. A test mimicking small intestinal conditions, however, is ideally started with an initial pH of about 6.5, the compositions being neutralised by addition of base in order to eliminate the effect of the acids.

A further object of the present invention was therefore to provide a composition (as described above) which allows efficient control of intestinal microflora at a fixed pH close to neutral conditions.

The range of relevant bacteria to be tested is wide and includes not only pathogens, but also some commensal bacteria. The reason is that at high enough concentration any acid inhibits the growth of any bacterium. The key question is whether or not the minimum inhibitory concentration for the pathogens is lower than for commensal intestinal bacteria. If the inhibitory concentration for beneficial bacteria is lower than for pathogens, the composition is not likely to be successful. In the opposite case, inclusion of the composition in the diet at lowest effective concentration would primarily improve the competitiveness of harmless commensal bacteria against the pathogens and lead to improved intestinal health for the host animal and safety for the end consumer.

A further object of the present invention was therefore to provide a composition (as described above) which is suitable to control intestinal microflora in production animals in such a way that growth of commensal bacteria is not inhibited and preferably providing beneficial commensal bacteria with a competitive advantage over pathogens.

In an extensive study it was now found out that a composition containing formic acid, propionic acid, one or more medium chain fatty acid(s) and one or more emulsifier(s) is particularly suitable to fulfill the above mentioned requirements.

The susceptibility of Salmonella enterica (pathogen), Clostridium perfringens (pathogen), Campylobacter jejuni (pathogen), Escherichia coli (pathogen/commensal) and lactobacillus crispatus (commensal) to the inhibitory effects of a number of compositions comprising organic acids and emulsifiers was tested at uncontrolled pH conditions as well as at a pH buffered to neutral.

It was found out that formic acid and propionic acid are the major drivers for the bacterial growth inhibition when pH is not controlled. This effect is most likely based on unspecific pH reduction. However, when the initial pH is controlled by neutralising the compositions, the components other than formic acid become essential drivers for bacterial growth inhibition and clearly boost the antibacterial effect.

The objectives of the present invention as set out above are met by a composition comprising or consisting of

(a) formic acid,

(b) propionic acid,

(c) one or more fatty acid(s) having 8 or 10 carbon atoms, preferably caprylic acid and/or capric acid, and

(d) one or more, preferably two, emulsifiers of the group of fatty acid esters, preferably glycerides, more preferably glycerol-monolaurate and/or ethoxylated castor oil.

A composition according to the invention suppresses the growth of bacterial pathogens under conditions, in which pH reduction by the acidity of the composition is not actively prevented and therefore allows to control pathogen contamination for example in animal feed already prior to feeding.

Under conditions where pH reduction is prevented, addition of one or more fatty acid(s) having 8 or 10 carbon atoms, in particular caprylic acid and/or capric acid, and one or more emulsifier(s) provides an antibacterially active system far superior to formic acid and propionic acid alone or combinations thereof. Therefore, when the digesta enters the intestinal tract distal to duodenum, where neutralisation occurs, a composition according to the invention efficiently retains its antibacterial activity. In case a combination of fatty acid(s) having 8 carbon atoms and fatty acid(s) having 10 carbon atoms is used, it is preferred that the ratio of C10:C8 is in the range of 40:60 to 60:40 (based on weight), preferably 50:50 or 40:60 or 60:40.

Furthermore, bacterial pathogens are far more sensitive to a composition according to the invention than commensal small intestinal bacteria providing a competitive advantage for beneficial and neutral bacteria over pathogens.

A composition according to the present invention is therefore suitable to efficiently control intestinal microflora in production animals prior to and during ingestion and to ensure animal health as well as safety for human consumers.

In a preferred embodiment of the invention, the total amount of component (a) is from 20 to 90 wt-%, preferably from 30 to 85 wt.-%, more preferably from 40 to 80 wt.-% based on the total weight of the composition.

In a further preferred embodiment of the present invention, the total amount of component (b) is from 1 to 50 wt-%, preferably from 2 to 40 wt.-%, more preferably from 5 to 30 wt.-% based on the total weight of the composition.

In a further preferred embodiment of the present invention, the total amount of component (c) is from 0.5 to 40 wt-%, preferably from 1 to 30 wt.-%, more preferably from 2 to 25 wt.-% based on the total weight of the composition.

In a further preferred embodiment of the present invention, the the total amount of component (d) is from 0.01 to 3 wt-%, preferably from 0.05 to 2.5 wt.-%, more preferably from 0.1 to 2 wt.-% based on the total weight of the composition.

The total amounts of the components (a) to (d) in the preferred embodiments are adjusted to provide a maximum antibacterial efficiency of the composition prior to feeding as well as in the intestinal tract of an animal.

According to a specific preferred embodiment of the present invention, component (c) is caprylic acid, wherein preferably

the total amount of component (a) is from 20 to 90 wt-%, preferably from 30 to 85 wt.-%, more preferably from 40 to 80 wt.-%, and/or

the total amount of component (b) is from 1 to 50 wt-%, preferably from 2 to 40 wt.-%, more preferably from 5 to 30 wt.-%, and/or

the total amount of component (c) is from 0.2 to 40 wt-%, preferably from 0.5 to 30 wt.-%, more preferably from 1 to 20 wt.-%, and/or

the total amount of component (d) is from 0.01 to 3 wt-%, preferably from 0.05 to 2.5 wt.-%, more preferably from 0.1 to 2 wt.-%,

in each case based on the total weight of the composition.

In the above mentioned studies, it has been found out, that caprylic acid is especially well suited to contribute to an efficient antibacterial composition when combined with the other components in the selected ranges as described above.

According to yet another specific preferred embodiment of the present invention, component (c) is a mixture of caprylic acid and capric acid, wherein preferably

the total amount of component (a) is from 20 to 90 wt-%, preferably from 30 to 85 wt.-%, more preferably from 40 to 80 wt.-%, and/or

the total amount of component (b) is from 1 to 50 wt-%, preferably from 2 to 40 wt.-%, more preferably from 5 to 30 wt.-%, and/or

the total amount of component (c) is from 1 to 50 wt-%, preferably from 2 to 40 wt.-%, more preferably from 3 to 30 wt.-%, and/or

the total amount of component (d) is from 0.01 to 3 wt-%, preferably from 0.05 to 2.5 wt.-%, more preferably from 0.1 to 2 wt.-%,

in each case based on the total weight of the composition.

In the above described studies, it has further been found out, that a mixture of caprylic acid and capric acid is also especially well suited to contribute to an efficient antibacterial composition when combined with the other components in the selected ranges as described above. Preferably the ratio of capric acid to caprylic acid in a composition according to the invention is in the range of 40:60 to 60:40 (by weight), preferably 50:50 or 40:60 or 60:40.

In a preferred embodiment of the present invention, the total amount of components (a) to (d) is >90 wt.-%, preferably >95 wt.-%, most preferably >99 wt.-% based on the total weight of the composition.

In order to ensure maximum antibacterial efficiency of the composition according to the invention, components (a) to (d) should be present in a sufficient amount as described above.

In one aspect the present invention also relates to a method for the production of a composition according to the invention, comprising the following steps:

(i) providing components (a), (b), (c) and (d)

(ii) mixing components (a), (b), (c) and (d).

All components (a) to (d) are commercially available or can be obtained by extraction from natural material or synthesis using methods known to a person skilled in the art.

A further aspect of the present invention relates to a composition as specified above for use in controlling intestinal microflora, in particular for reducing foodborne pathogens, in production animals, in particular poultry and pigs.

A composition according to the invention has been shown to be suitable to control intestinal microflora in a way that growth of pathogens is suppressed while growth of neutral or beneficial bacteria is not inhibited. Health and productivity of the animals as well as safety for human consumers is therefore increased when a composition according to the invention is used to supplement animal feed.

According to a preferred embodiment, the foodborne pathogens are selected from the group consisting of Salmonella enterica, Clostridium perfringens, Campylobacter jejuni and Escherichia coli.

The composition according to the invention has been shown to have strong inhibiting effects on the growth of common foodborne human pathogens such as the ones listed above. A possible application of a composition according to the invention, however, is not limited to these species but may include others.

In a further aspect, the present invention also relates to an animal feed or semi-finished product for preparing animal feed, comprising a composition according to the invention.

In the context of the present invention the term “animal feed” relates to all forms of animal diet of solid or liquid nature, including e.g. water based drinks.

When used to supplement production animal diet, a composition according to the invention is suitable to prevent pathogen contamination of animal feed and drink already prior to feeding as well as to control intestinal microflora in the production animal.

In a preferred embodiment of the present aspect of the invention, the total amount of the composition is in the range of from 0.1 wt.-% to 3.0 wt.-% based on the total weight of the feed.

The invention further relates to a semi-finished product, in particular in form of a concentrate, wherein the total amount of the composition as described above is 10 wt.-% or more, preferably 30 wt.-% or more, in particular 50 wt.-% or more, or wherein the product consists of the composition

To ensure maximum efficiency of the feed supplementation, the composition according to the invention should be present in a sufficient amount as described above.

The following examples are added to illustrate the antibacterial activity of compositions according to the invention.

### EXAMPLE COMPOSITIONS

### Content in g/kg Compostion

| | com- | com- | comparative | comparative |
|---|---|---|---|---|
| | position A | position B | composition C | composition D |
| formic acid | 694 | 694 | 1000 | 700 |
| propionic | 200 | 200 | — | 200 |
| acid | | | | |
| caprylic acid | 100 | — | — | — |
| capric acid/ | — | 100 | — | 100 |
| caprylic acid | | | | |
| 40:60 (by | | | | |
| weight) | | | | |
| glycerol | 3 | 3 | — | — |
| monolaurate | | | | |
| ethoxylated | 3 | 3 | — | — |
| castor oil | | | | |

The susceptibility of the following bacteria was tested:

1. Salmonella enterica (pathogen)

2. Clostridium perfringens (pathogen)

3. Campylobacter jejuni (pathogen)

4. Escherichia coli (pathogen/commensal)

5. Lactobacillus crispatus (commensal)

Culture media:

S. enterica, Cl. perfringens, C. jejuni and E. coli were grown in Tryptic soy broth with glucose and yeast extract (TSGY—medium). L. crispatus was grown on de Man, Rogosa and Sharpe medium (MRS). For S. enterica, C. jejuni and E. coli the medium was further amended with 100 mM phosphate buffer and for Cl. perfringens with 50 mM phosphate.

pH Control During the Test:

The test mimicking feed preservation application was carried out in a medium the pH of which was allowed to shift freely when acids at various concentration were added. The test mimicking small intestinal conditions was started at about pH 6.5 with acid compositions neutralised with 32% NaOH. This test measured the effect of the acids under the conditions where pH effect was eliminated.

Inoculation and Growth Conditions:

S. enterica and E. coli were inoculated with 1% inoculum whereas with C. jejuni, Cl. perfringens and L. crispatus 5% inoculum was used. Cultures were grown under anaerobic conditions at 40° C. for a period of time that depended on the bacterium in question; nine hours for S. enterica and E. coli, 19 h for C. jejuni and 23 h for Cl. perfringens and L. crispatus. The results are expressed as percentage of the final growth of the control with no test composition added.

Monitoring of Growth:

The density of bacterial suspensions was measured at 0 h and again after the indicated growth time. The signal at 0 h makes it possible to estimate the strength of inoculum as compared to the growth in the end of the culturing. For all bacteria other than C. jejuni the growth was recorded by measuring turbidity at the wavelength of 660 nm. With C. jejuni the bacterial density in the culture was measured by using SYBR Green, a fluorescing dye which binds to DNA in bacterial cells. The DNA-dye-complex absorbs blue light (λmax=497 nm) and emits green light (λmax=520 nm). The latter was quantified by a fluorometer. C. jejuni cultures were fixed with 8% glutardialdehyde and refrigerated overnight. 500 μl of bacterial suspension was centrifuged at 17 000×g for 10 minutes, bacteria resuspended in TE buffer, centrifuged again and the pellet suspended in 125 μl of the buffer. The bacterial suspension was mixed 1:1 with 1:5000 diluted SYBR Green solution and the fluorescence measured.

Treatments:

All tested compositions were introduced in bacterial growth media at three concentrations, 0.01, 0.1 and 0.3% (w/v). Also, each test composition and concentration was introduced at free pH and at pH adjusted to 6.5.

Growth of Indicator Bacteria (% of Growth Control) at Free pH:

| | Composition A | | | Composition B | | | Composition C | | | Composition D | | |
|---|---|---|---|---|---|---|---|---|---|---|---|---|
| | 0.01% | 0.1% | 0.3% | 0.01% | 0.1% | 0.3% | 0.01% | 0.1% | 0.3% | 0.01% | 0.1% | 0.3% |
| E. coli | 85 | 15 | 5 | 90 | 0 | 20 | 80 | 20 | 15 | 100 | 30 | 35 |
| S. enterica | 95 | 20 | 5 | 95 | 0 | 10 | 80 | 25 | 20 | 130 | 35 | 40 |
| C. perfringens | 55 | 30 | 25 | 60 | 20 | 25 | 75 | 20 | 15 | 110 | 30 | 30 |
| C. jejuni | 110 | 10 | 10 | 105 | 10 | 10 | 170 | 20 | 10 | 150 | 25 | 10 |
| L. crispatus | 105 | 80 | 60 | 90 | 50 | 50 | 95 | 90 | 75 | 100 | 95 | 120 |

Growth of Indicator Bacteria (% of Growth Control) at pH 6.5

| | Composition A | | | Composition B | | | Composition C | | | Composition D | | |
|---|---|---|---|---|---|---|---|---|---|---|---|---|
| | 0.01% | 0.1% | 0.3% | 0.01% | 0.1% | 0.3% | 0.01% | 0.1% | 0.3% | 0.01% | 0.1% | 0.3% |
| E. coli | 100 | 55 | 15 | 100 | 50 | 15 | 110 | 55 | 45 | 105 | 55 | 45 |
| S. enterica | 105 | 55 | 15 | 105 | 50 | 20 | 90 | 55 | 50 | 115 | 60 | 50 |
| C. perfringens | 100 | 75 | 30 | 45 | 70 | 20 | 100 | 65 | 60 | 65 | 40 | 60 |
| C. jejuni | 120 | 60 | 15 | 85 | 20 | 10 | 170 | 115 | 65 | 120 | 70 | 15 |
| L. crispatus | 110 | 105 | 90 | 100 | 95 | 70 | 120 | 115 | 95 | 105 | 115 | 125 |

The results show that the compositions A and B are able to suppress growth of the test bacteria more efficiently, especially at pH 6.5, than pure formic acid (composition C) or a combination of formic acid and medium chain fatty acids without emulsifier(s) (composition D). At the same time, growth of commensal l. crispatus is barely suppressed by compositions A and B, allowing the beneficial bacterium to gain competitive advantage over the pathogens.

1.-14. (canceled)

15. A composition comprising
(a) formic acid,
(b) propionic acid,
(c) one or more fatty acid(s) having 8 or 10 carbon atoms, and
(d) one or more emulsifiers of the group of fatty acid esters.

16. The composition of claim 15, wherein the total amount of component (a) is from 20 to 90 wt-%, based on the total weight of the composition.

17. The composition of claim 15, wherein the total amount of component (b) is from 1 to 50 wt-%, based on the total weight of the composition.

18. The composition of claim 15, wherein the total amount of component (c) is from 0.5 to 40 wt-%, based on the total weight of the composition.

19. The composition according claim 15, wherein the total amount of component (d) is from 0.01 to 3 wt-%, based on the total weight of the composition.

20. The composition of claim 15, wherein component (c) is caprylic acid and
the total amount of component (a) is from 20 to 90 wt-%,
the total amount of component (b) is from 1 to 50 wt-%,
the total amount of component (c) is from 0.2 to 40 wt-%, and
the total amount of component (d) is from 0.01 to 3 wt-%,
in each case based on the total weight of the composition.

21. The composition of claim 15, wherein component (c) is a mixture of caprylic acid and capric acid and
the total amount of component (a) is from 20 to 90 wt-%,
the total amount of component (b) is from 1 to 50 wt-%,
the total amount of component (c) is from 1 to 50 wt-%, and
the total amount of component (d) is from 0.01 to 3 wt-%,
in each case based on the total weight of the composition.

22. The composition of claim 15, wherein the total amount of components (a) to (d) is >90 wt.-%, based on the total weight of the composition.

23. A method for the production of the composition of claim 15, said method comprising:
(i) providing components (a), (b), (c) and (d); and
(ii) mixing components (a), (b), (c) and (d).

24. The composition of claim 15 for use in controlling intestinal microflora, in particular for reducing foodborne pathogens, in production animals, in particular poultry.

25. The composition of claim 24, wherein the foodborne pathogens are selected from the group consisting of Salmonella enterica, Clostridium perfringens, Campylobacter jejuni, Escherichia coli.

26. Animal feed comprising the composition of claim 15.

27. The animal feed of claim 27, wherein the total amount of the composition is in the range of from 0.1 wt.-% to 3.0 wt.-% based on the total weight of the feed.

28. A semi-finished product for preparing animal feed comprising the composition of claim 15, wherein the total amount of the composition is 10 wt.-% or more.

29. A semi-finished product for preparing animal feed consisting of the composition of claim 15.

30. The composition of claim 15, wherein the composition consists of
(a) formic acid,
(b) propionic acid,
(c) one or more fatty acid(s) having 8 or 10 carbon atoms, preferably caprylic acid and/or capric acid, and
(d) one or more, preferably two, emulsifiers of the group of fatty acid esters, preferably glycerides, more preferably glycerol-monolaurate and/or ethoxylated castor oil.

31. The composition of claim 30, wherein the total amount of component (a) is from 20 to 90 wt-%, based on the total weight of the composition.

32. The composition of claim 30, wherein the total amount of component (b) is from 1 to 50 wt-%, based on the total weight of the composition.

33. The composition of claim 30, wherein the total amount of component (c) is from 0.5 to 40 wt-%, based on the total weight of the composition.

34. The composition according claim 30, wherein the total amount of component (d) is from 0.01 to 3 wt-%, based on the total weight of the composition.
